(19) United States
(12) Patent Application Publication
(10) Pub. No.: US 2014/0350050 A1
(43) Pub. Date: Nov. 27, 2014

(54) PYRIDINE COMPOUNDS AS INHIBITORS OF KINASE

(71) Applicant: TELIGENE LTD., Suzhou (CN)

(72) Inventor: Dawei Zhang, Thousand Oaks, CA (US)

(21) Appl. No.: 14/345,400

(22) PCT Filed: Sep. 20, 2012
(86) PCT No.: PCT/CN2012/081694

§ 371 Date: Mar. 17, 2014

Related U.S. Application Data
(60) Provisional application No. 61/626,104, filed on Sep. 21, 2011.

(51) Int. Cl. C07D 401/14; A61K 31/4545; A61K 45/06

(52) U.S. Cl. CPC C07D 401/14; A61K 45/06; A61K 31/4545
USPC 514/318; 546/194

(57) ABSTRACT

The present invention relates to novel pyridines, their derivatives, pharmaceutically acceptable salts, solvates, entiomers, prodrugs and hydrates thereof. The compounds and compositions of the present invention have protein kinases inhibitory activities and are expected to be useful for the treatment of protein kinases mediated diseases and conditions, including hyper-proliferative disorder, ALK kinases mediated disorder, and neoplasia.

### CROSS REFERENCE

This invention claims the benefit of U.S. Provisional Patent Application No. 61/626,104, filed on Sep. 21, 2011, which is incorporated herein by reference.

### FIELD OF THE INVENTION

The present invention relates to inhibitors of kinase and pharmaceutically acceptable salts, solvates, hydrates, prodrugs and metabolites thereof, the preparation method thereof, and the use of such compounds to treat kinase mediated diseases and conditions such as cancer.

### BACKGROUND OF THE INVENTION

Protein kinases represent a large family of enzymes, which catalyze the phosphorylation of target protein substrates. The phosphorylation is usually a transfer reaction of a phosphate group from ATP to the protein substrate. Common points of attachment for the phosphate group to the protein substrate include, for example, a tyrosine, serine or threonine residue. Examples of kinases in the protein kinase family include, without limitation, Abl1 (v-Abl Abelson murine leukemia viral oncogene homolog 1), Akt, Alk, Bcr-Abl1, Blk, Brk, Btk, c-Kit, c-Met, c-Src, c-Fms, CDK1-10, b-Raf, c-Raf1, CSF1R, CSK, EGFR, ErbB2, ErbB3, ErbB4, Erk, FGFR1, FGFR2, FGFR3, FGFR4, FGFR5, Flt-1, Fps, Frk, Jak, KDR, MEK, PDGFR, PIK, PKC, PYK2, Ros, Tie, Tie2, and Zap70. Due to their activity in numerous cellular processes, protein kinases have emerged as important therapeutic targets.

ALK (Anaplastic Lymphoma Kinase) is a 1620 amino acid transmembrane protein, consisting of extracellular domain with amino-terminal signal peptide, intracellular domain with a juxtamembranous segment harboring a binding site for insulin receptor substrate-1, and a carboxy-terminal kinase domain. ALK is a member of the insulin receptor tyrosine kinases, Echinoderm microtubule-associated protein-like 4 (EML4) is a 120 KDa cytoplasmic protein, which involves in the formation of microtubules and microtubule binding protein. EML4-ALK is a novel fusion gene arising from an inversion on the short arm of chromosome 2 that joined exons 1-13 of EML4 to exons 20-29 of ALK. The presence of EML4-ALK fusion is identified in approximately 3-13% of NSCLC (non-small cell lung cancer) patients.

c-Met (MET or MNNG HOS Transforming gene) is a proto-oncogene that encodes a protein known as hepatocyte growth factor receptor (HGFR). The hepatocyte growth factor receptor protein possesses tyrosine-kinase activity. The primary single chain precursor protein is post-translationally cleaved to produce the alpha and beta subunits, which are disulfide linked to form the mature receptor. Abnormal MET activation in cancer correlates with poor prognosis, where aberrantly active MET triggers tumor growth, formation of new blood vessels (angiogenesis) that supply the tumor with nutrients, and cancer spread to other organs (metastasis). MET is deregulated in many types of human malignancies, including cancers of kidney, liver, stomach, breast, and brain.

To this end, attempts have been made to identify small molecules which act as PK inhibitors. For example, Amino heteroaryl compounds diaryl ureas (PCT WO2004/076412) have been described as ALK/c-MET inhibitors. Azaindole derivatives (PCT WO2010/068292) have been described as ALK/EGFR kinase inhibitors.

Thus, the compounds that can inhibit protein kinases such as ALK and other kinases activity either independently or together can be used to treat human diseases such as Cancers.

### SUMMARY OF THE INVENTION

The present invention provides compounds of Formula I:

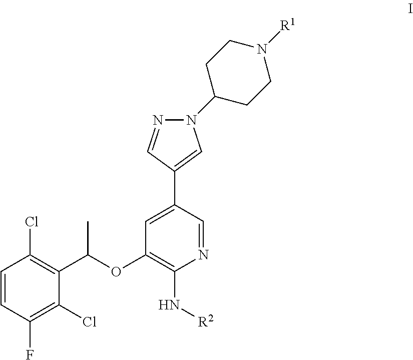

or a pharmaceutically acceptable salt, solvate or a prodrug or an enantiomer, or a metabolite thereof, wherein

R¹ is hydrogen, substituted or unsubstituted C₁₋₆ alkyl;

R² is hydrogen, substituted or unsubstituted C₁₋₆ alkyl, —C(O)R³ or —C(O)OR³,

R³ is substituted or unsubstituted C₁₋₆ alkyl;

with the proviso that R¹ and R² are not both hydrogen.

The present invention further provides pharmaceutical compositions comprising a compound of formula I described above and a pharmaceutically acceptable carrier.

The present invention further provides methods for regulating the kinase signaling transduction comprising administrating to a mammalian subject a therapeutically effective amount of any of the compounds of formula I described above.

### DETAILED DESCRIPTION OF THE INVENTION

In some embodiments of the present invention, there are provided compounds of Formula I:

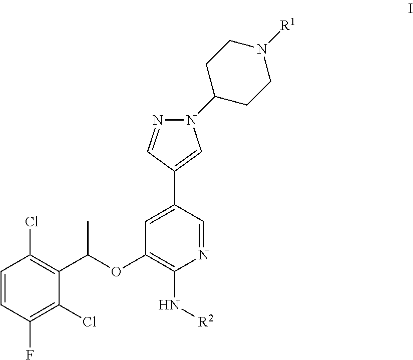

or a pharmaceutically acceptable salt, solvate or an enantiomer, or a prodrug or a metabolite thereof, wherein

R¹ is hydrogen, substituted or unsubstituted C₁₋₆ alkyl;

R² is hydrogen, substituted or unsubstituted C₁₋₆ alkyl, —C(O)R³ or —C(O)OR³,

R³ is substituted or unsubstituted C₁₋₆ alkyl;

with the proviso that R¹ and R² are not both hydrogen, and R¹ is substituted or unsubstituted deuterated C₁₋₆ alkyl when R² is hydrogen.

In certain embodiments, R¹ is hydrogen, and R² is C₁₋₄ alkyl, or C₁₋₄ acyl. In other embodiments, R¹ is a methyl or ethyl, and R² is C₁₋₄ alkyl, or C₁₋₄ acyl. In other embodiments, R¹ is CD₃ or CD₂CD₃, and R² is hydrogen, C₁₋₄ alkyl, or C₁₋₄ acyl. In preferred embodiments, R¹ is CD₃ or CD₂CD₃, and R² is hydrogen. In preferred embodiments, R¹ is hydrogen, methyl or ethyl, and R² is —C(O)CH₃ (i.e. acetyl). In preferred embodiments, R¹ is hydrogen, methyl or ethyl, and R² is —C(O)OCH₂CH₃.

In certain embodiments, there are provided compounds without limitation selected from the group consisting of:
3-(1-(2,6-dichloro-3-fluorophenyl)ethoxy)-5-(1-(1-ethylpiperidin-4-yl)-1H-pyrazol-4-yl)pyridin-2-amine,
N-(3-(1-(2,6-dichloro-3-fluorophenyl)ethoxy)-5-(1-(piperidin-4-yl)-1H-pyrazol-4-yl)pyridin-2-yl)acetamide,
N-(3-(1-(2,6-dichloro-3-fluorophenyl)ethoxy)-5-(1-(1-methylpiperidin-4-yl)-1H-pyrazol-4-yl)pyridin-2-yl)acetamide;
N-(3-(1-(2,6-dichloro-3-fluorophenyl)ethoxy)-5-(1-(1-ethylpiperidin-4-yl)-1H-pyrazol-4-yl)pyridin-2-yl)acetamide;
3-(1-(2,6-dichloro-3-fluorophenyl)ethoxy)-N-ethyl-5-(1-(piperidin-4-yl)-1H-pyrazol-4-yl)pyridin-2-amine;
3-(1-(2,6-dichloro-3-fluorophenyl)ethoxy)-5-(1-(piperidin-4-yl)-1H-pyrazol-4-yl)-N-propylpyridin-2-amine;
3-(1-(2,6-dichloro-3-fluorophenyl)ethoxy)-5-(1-(1-d₃-methyl-piperidin-4-yl)-1H-pyrazol-4-yl)pyridin-2-amine;
3-(1-(2,6-dichloro-3-fluorophenyl)ethoxy)-5-(1-(1-d₅-ethyl-piperidin-4-yl)-1H-pyrazol-4-yl)pyridin-2-amine,
(S)—N-(3-(1-(2,6-dichloro-3-fluorophenyl)ethoxy)-5-(1-(piperidin-4-yl)-1H-pyrazol-4-yl)pyridin-2-yl)acetamide,
(S)-3-(1-(2,6-dichloro-3-fluorophenyl)ethoxy)-5-(1-(1-methylpiperidin-4-yl)-1H-pyrazol-4-yl)pyridin-2-amine;
(S)-3-(1-(2,6-dichloro-3-fluorophenyl)ethoxy)-5-(1-(1-d₃-methyl-piperidin-4-yl)-1H-pyrazol-4-yl)pyridin-2-amine,
(S)-3-(1-(2,6-dichloro-3-fluorophenyl)ethoxy)-5-(1-(1-ethylpiperidin-4-yl)-1H-pyrazol-4-yl)pyridin-2-amine,
(S)-3-(1-(2,6-dichloro-3-fluorophenyl)ethoxy)-5-(1-(1-d₅-ethyl-piperidin-4-yl)-1H-pyrazol-4-yl)pyridin-2-amine,
(R)—N-(3-(1-(2,6-dichloro-3-fluorophenyl)ethoxy)-5-(1-(piperidin-4-yl)-1H-pyrazol-4-yl)pyridin-2-yl)acetamide,
(R)-3-(1-(2,6-dichloro-3-fluorophenyl)ethoxy)-5-(1-(1-d₃-methyl-piperidin-4-yl)-1H-pyrazol-4-yl)pyridin-2-amine,
(R)-3-(1-(2,6-dichloro-3-fluorophenyl)ethoxy)-5-(1-(1-d₅-ethyl-piperidin-4-yl)-1H-pyrazol-4-yl)pyridin-2-amine, and
(R)-3-(1-(2,6-dichloro-3-fluorophenyl)ethoxy)-5-(1-(1-ethylpiperidin-4-yl)-1H-pyrazol-4-yl)pyridin-2-amine,
and the like, or a pharmaceutically acceptable salt, solvate, or a prodrug, or a metabolite thereof.

In some embodiments, the compound of this invention is an enantiomer. In other embodiments, the compound of this invention is a diastereomer. In another embodiment, the deuterium enrichment in compounds of this invention is at least about 1%.

In some embodiments, the present invention provides pharmaceutical compositions comprising a compound of the invention and a pharmaceutically acceptable carrier. In certain embodiments, the compositions are for the treatment of a disease regulated by a protein kinase. In certain embodiments, the compositions are for the treatment of a hyper-proliferative disorder and/or angiogenesis disorder. In some embodiments, the pharmaceutical compositions further comprise an anti-neoplastic agent, an immunosuppressant, an immunostimulant, or combination thereof. In other embodiments, the pharmaceutical compositions are suitable for oral, parenteral, or intravenous administration.

In some embodiments, the present invention provides methods for regulating the kinase signaling transduction comprising administrating to a mammalian subject a therapeutically effective amount of any of the inventive compounds described herein.

In other embodiments, the present invention provides herein methods for treating or preventing a ALK (including all fusion and/or mutant kinases), c-Met mediated disorder, said method comprises administrating to a mammalian subject a therapeutically effective amount of any of the inventive compounds described herein.

In yet another aspect, there are provided herein methods for inhibiting both ALK (including all fusion and/or mutant kinases) and c-Met kinases, said method comprises administrating to a mammalian subject a therapeutically effective amount of any of the inventive compounds described herein.

In other embodiments, the present invention provides methods for treating neoplasia comprising administrating to a mammalian subject in need thereof, a therapeutically effective amount of any of the inventive compounds described herein. In certain embodiments, the neoplasia is selected from skin cancer, leukemias, colon carcinoma, renal cell carcinoma, gastrointestinal stromal cancer, solid tumor cancer, myeloma, breast cancer, pancreatic carcinoma, non-small cell lung cancer, non-hodgkin's lymphoma, hepatocellular carcinoma, thyroid cancer, bladder cancer, colorectal cancer, and prostate cancer. In some embodiments, the methods further comprises administering one or more anti-cancer agents.

In other embodiments, there are provided methods for treating or preventing a hyper-proliferative and/or angiogenesis comprising administrating to a mammalian subject a therapeutically effective amount of any of the inventive compounds described herein.

The following definitions should assist in understanding the invention described herein.

The term “alkyl” is intended to include linear, branched, cyclic hydrocarbon group, which may be unsubstituted or optionally substituted with one or more functional groups. C₁-C₆ alkyl is intended to include C₁, C₂, C₃, C₄, C₅ and C₆ alkyl groups. Examples of alkyl include, but not limited to, methyl, ethyl, n-propyl, isopropyl, cyclopropyl, etc. Alkyl may be substituted or unsubstituted. Illustrative substituted alkyl group include, but are not limited to, fluoromethyl, difluoromethyl, trifluoromethyl, hydoxymethyl, benzyl, etc.

Halogen means fluorine, chlorine, bromine, and iodine.

The invention also includes isotopically-labeled compounds of the invention, wherein one or more atoms is replaced by an atom having the same atomic number, but an atomic mass or mass number different from the atomic mass or mass number usually found in nature. Examples of isotopes suitable for inclusion in the compounds of the invention include isotopes of hydrogen, such as Deuterium and carbon such as ¹³C.

Deuterium (D or ²H) is a non-radioactive, stable isotope of hydrogen, the natural abundance of deuterium is 0.015%. Compound should be considered to be unnatural, if its level of deuterium has been enriched to be greater than their natural abundance level 0.015%. In a compound of this invention, it is understood that the abundance of deuterium is substantially greater than the natural abundance of deuterium, which is 0.015%, when a particular position is designated as deuterium. A position designated as deuterium typically has a minimum isotopic enrichment factor of at least 3000 at each atom designated as deuterium in said compound. The concentration of naturally abundant stable hydrogen is small and immaterial compared to the degree of stable isotopic substitution of compounds of this invention.

The term “pharmaceutically acceptable” when used with reference to a compound of the invention is intended to refer to a form of the compound that is safe for administrating to a subject. For example, a free base, a salt form, a solvate, a hydrate, a prodrug or derivative form of a compound of this invention, which has been approved for mammalian use, via oral ingestion or any other route of administration, by a governing authority or regulatory agency, such as the Food and Drug Administration (FDA) of the United States, is pharmaceutically acceptable.

The phrase “effective amount” is intended to quantify the amount of each agent, which will achieve the goal of improvement in disorder severity and the frequency of incidence over treatment of each agent by itself, while avoiding adverse side effects typically associated with alternative therapies. The effective amount, in one embodiment, is administered in a single dosage form or in multiple dosage forms.

The compounds of this invention in some embodiments also are represented in multiple tautomeric forms. The invention includes all tautomeric forms of the compounds described herein.

The compounds in one embodiment also have cis- or trans- or E- or Z-double bond isomeric forms. All such isomeric forms of such compounds are included in the present invention.

The present invention provides compounds which are capable of modulating one or more signal transduction pathways comprising, but not limited to ALK and/or cMet kinase.

By the term “modulate,” it is meant that the functional activity of the pathway (or a component of it) is changed in comparison to its normal activity in the absence of the compound. This effect includes any quality or degree of modulation, including, increasing, agonizing, augmenting, enhancing, facilitating, stimulating, decreasing, blocking, inhibiting, reducing, diminishing, antagonizing, etc.

The compounds of the present invention can also modulate one or more of the following processes, including, but not limited to, e.g., cell growth (including, e.g., differentiation, cell survival, and/or proliferation), tumor cell growth (including, e.g., differentiation, cell survival, and/or proliferation), tumor regression, endothelial cell growth (including, e.g., differentiation, cell survival, and/or proliferation), angiogenesis (blood vessel growth), lymphangiogenesis (lymphatic vessel growth), and/or hematopoiesis (e.g., T- and B-cell development, dendritic cell development, etc.).

While not wishing to be bound by any theory or mechanism of action, it has been found that compounds of the present invention possess the ability to modulate kinase activity. The methods of the present invention, however, are not limited to any particular mechanism or how the compounds achieve their therapeutic effect. By the phrase “kinase activity,” it is meant a catalytic activity in which a gamma-phosphate from adenosine triphosphate (ATP) is transferred to an amino acid residue (e.g., serine, threonine, or tyrosine) in a protein substrate. A compound can modulate kinase activity, e.g., inhibiting it by directly competing with ATP for the ATP-binding pocket of the kinase, by producing a conformational change in the enzyme's structure that affects its activity (e.g., by disrupting the biologically-active three-dimensional structure), by binding to and locking the kinase in an inactive conformation, etc.

Formulations and Method of Use

The amount of compound(s) which is/are administered and the dosage regimen for treating cancer with the compounds and/or compositions of this invention depends on a variety of factors, including the age, weight, sex and medical condition of the subject, the type of disease, the severity of the disease, the route and frequency of administration, and the particular compound employed. Thus, the dosage regimen may vary widely, but can be determined routinely using standard methods. A daily dose of about 0.01 to 500 mg/kg, advantageously between about 0.01 and about 50 mg/kg, more advantageously about 0.01 and about 30 mg/kg, even more advantageously between about 0.1 and about 10 mg/kg may be appropriate, and should be useful for all methods of use disclosed herein. The daily dose can be administered in one to four doses per day.

While it may be possible to administer a compound of the invention alone, in the methods described, the compound administered normally will be present as an active ingredient in a pharmaceutical composition. Thus, in another embodiment of the invention, there is provided a pharmaceutical composition comprising a compound of this invention in combination with a pharmaceutically acceptable carrier, which includes diluents, excipients, adjuvants and the like (collectively referred to herein as “carrier” materials) as described herein, and, if desired, other active ingredients. A pharmaceutical composition of the invention may comprise an effective amount of a compound of the invention or an effective dosage amount of a compound of the invention. An effective dosage amount of a compound of the invention includes an amount less than, equal to or greater than an effective amount of the compound; for example, a pharmaceutical composition in which two or more unit dosages, such as in tablets, capsules and the like, are required to administer an effective amount of the compound, or alternatively, a multi-dose pharmaceutical composition, such as powders, liquids and the like, in which an effective amount of the compound is administered by administering a portion of the composition.

Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nanoparticulates, gels, solid solution, liposome, films (including muco-adhesive), ovules, sprays and liquid formulations.

The compounds of the invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and PGLA microspheres.

Combinations

While the compounds of the invention can be dosed or administered as the sole active pharmaceutical agent, they can also be used in combination with one or more compounds of the invention or in conjunction with other agents. When administered as a combination, the therapeutic agents can be formulated as separate compositions that are administered simultaneously or sequentially at different times, or the therapeutic agents can be given as a single composition.

Biological Assays:

As stated hereinbefore, the compounds defined in the present invention possess anti-proliferation activity. These properties may be assesses, for example, using one or more of the procedures set out below:

An in vitro assay which determines the ability of a test compound to inhibit ALK kinase.

a) kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). All reactions were performed in polystyrene 96-well plates in a final volume of 0.135 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR.

b) An alternative method to conduct the kinase assay with the following condition and procedure: Reagent: Base Reaction buffer; 20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO.

Reaction Procedure:

1. Preparing indicated substrate in freshly prepared Base Reaction Buffer

2. Delivering any required cofactors to the substrate solution above

3. Delivering indicated kinase into the substrate solution and gently mix

4. Delivering compounds in DMSO into the kinase reaction mixture

5. Delivering ³³P-ATP (specific activity 0.01 μCi/μl final) into the reaction mixture to initiate the reaction.

6. Incubating kinase reaction for 120 min. at room temperature

7. Reactions are spotted onto P81 ion exchange paper (Whatman #3698-915)

8. Washing filters extensively in 0.75% Phosphoric acid.

Compounds were tested in 10-dose IC50 mode with 3-fold serial dilution starting at 10 μM; Reactions were carried out at 10 μM ATP.

c) An alternative method to conduct the kinase assay with the following condition and procedure: The assay was performed using Kinase-Glo Plus luminescence kinase assay kit (Promega). It measures kinase activity by quantitating the amount of ATP remaining in solution following a kinase reaction. The luminescent signal from the assay is correlated with the amount of ATP present and is inversely correlated with the amount of kinase activity.

The compound were diluted in 10% DMSO and 5 μl of the dilution was added to a 50 μl reaction so that the final concentration of DMSO is 1% in all of reactions. All of the enzymatic reactions were conducted at 30° C. for 40 minutes. The 50 μl reaction mixture contains 40 mM Tris, pH 7.4, 10 mM MgCl₂, 0.1 mg/ml BSA, 1 mM DTT, 0.2 mg/ml substrate peptide, 10 μM ATP and ALK (Table 2.3.1). After the enzymatic reaction, 50 μl of Kinase-Glo Plus Luminescence kinase assay solution (Promega) was added to each reaction and incubate the plate for 5 minutes at room temperature. Luminescence signal was measured using a BioTek Synergy 2 microplate reader. ALK activity assays were performed in duplicate at each concentration. The luminescence data were analyzed using the computer software, Graphpad Prism. The difference between luminescence intensities in the absence of ALK (Luₜ) and in the presence of ALK (Lu_{c}) was defined as 100% activity (Luₜ−Lu_{c}). Using luminescence signal (Lu) in the presence of the compound, % activity was calculated as % activity={(Luₜ−Lu)/(Luₜ−Lu_{c})}×100%, where Lu=the luminescence intensity in the presence of the compound (all percent activities below zero were shown zero in the table). The values of % activity versus a series of compound concentrations were then plotted using non-linear regression analysis of Sigmoidal dose-response curve generated with the equation Y=B+(T−B)/1+10^{((Log EC50-X)×Hill Slope)}, where Y=percent activity, B=minimum percent activity, T=maximum percent activity, X=logarithm of compound and Hill Slope=slope factor or Hill coefficient. The IC₅₀ value was determined by the concentration causing a half-maximal percent activity.

BAF3-EML4-ALK Cell Assay:

A method of establishing BAF3-EML4-ALK stable cell line: Transfer 5×10⁶ BAF3 cells to a new tube, pellet the cells, and remove PBS by aspiration.; Resuspend the cells in 1 ml electroporation buffer, this provides 5×10⁶ of cells; Transfer 200 μl of cell suspension to EP tube, add 5 ug DNA, flip and place it on ice for 10 min; Pipet the 200 μl cell suspension into 2 mm electroporation cuvette; Place the cuvette into shockpod, close the lid, and press the PULSE button; Set parameters: Expentional decay pulse: VOL=200 V; CAP=900 uF, RES=1000Ω; Transfer all cells from electroporation cuvette to 6 well plate, adding 2 ml DMEMF12 containing 5 ng/ml IL3, 24 h later, replace the medium with DMEM F12 containing 2 μg/ml blasticidin S and 5 ng/ml IL3, maintain for 3 days; change the medium with DMEM F12 containing 2 ug/ml blasticidin S and 2.5 ng/ml IL3, maintain 3 days; change the medium with DMEM F12 containing 2 ug/Ml blasticidin S, maintain to culture.

MTT Cell proliferation assay: 5×10³ per well cell with 100 μl medium was seeded in 96-well plate; 24 hours later, 100 μl fresh medium with various concentration of compounds; After the cells were treated by compounds for 72 hours, add 20 μl MTT (5 mg/ml) into each wells; The plate is incubated at 37° C. for 4 hours; Centrifuge the plate at 800 g for 10 mins, Aspirated the medium, add 150 μl DMSO into each well. Shake plates gently for 10 mins, Measure the absorbance at 570 nm on plate reader; IR=(WC−WT)/WC.

The following TABLE A lists compounds representative of the invention and their activity in ALK kinase assay and BAF3-EML4-ALK Cell assay. In these assays, the following grading was used: I≦1 μM, 1 μM>II>0.1 μM, and III≦0.1 μM for IC₅₀.

TABLE A

| Compound No. | ALK | BAF3-EML4-ALK Cell |
|---|---|---|
| 7 | III | III |
| 9 | III | III |
| 10 | II | II |
| 11 | I | I |
| 12 | I | I |
| 13 | II | I |
| 14 | II | I |
| 15 | II | I |
| 16 | II | I |
| 17 | II | I |
| 18 | III | III |
| 19 | III | III |
| 20 | III | III |
| 21 | III | III |
| 22 | III | III |

For example, IC₅₀ of compounds 20 is 2.60 nM and IC₅₀ of compound 22 is 1.35 nM when they were tested in the ALK kinase assay.

A representative protocol for the in vivo experiment is as followed to establish the subcutaneous EML4-ALK-BAF3 cell line xenograft model in nude mice and to evaluate the in vivo therapeutic efficacy of the compounds: Animals: Male Balb/c nude mice (6˜8 weeks old) were obtained from SLAC Laboratory Animal, Shanghai, China. Animals were maintained under SPF conditions in sterile filter top cages and housed on HEPA-filtered ventilated racks. Animals received sterile rodent chow and water ad libitum. Cell line: BAF3-EML4-ALK stable cell line, the BAF3 cell which could expressed fusion oncogene EML4-ALK. S.c. Xenograft Models in Athymic Mice: Cells for implantation into athymic mice were harvested and pelleted by centrifugation at 1200 r/min for 5 min. Cells were washed once and resuspended in sterile PBS buffer with 5×10⁶ in 200 μl. Then cells were implanted s.c. into the right scapular region of each mouse and allowed to grow to 200˜300 mm³ before the administration of compound. Preparation of the Dose Formulation: each compound was suspensioned in 0.5% CMC-Na. Randomization: When tumor volumes approach 200˜300 mm³, the mice will be randomized into 5 groups according to the tumor volume. The day will be denoted as D1 and the treatments will be started at this day. Administered: Dose will be administered with oral gavage needle once daily for number of days. Treatment of compounds administered in 0.5% CMC-Na by p.o. gavage was initiated when tumors were 200˜300 mm³ in volume. Observations: After inoculation, the animals will be checked daily for morbidity and mortality. At the time of routine monitoring, the animals will be checked for any effects of tumor growth and treatments on normal behavior such as mobility, body weight gain/loss (body weights will be measured twice weekly or every other day), eye/hair matting and any other abnormal effect. Death and observed clinical signs will be recorded on the basis of the numbers of animals within each subset. Tumor Size Measurements: Tumor volume was determined by measurement with electronic vernier calipers every 3 days and tumor volume was calculated as the product of its length×width²×0.5. Effect studies: Tumor volume was expressed on indicated days as the mean tumor volume ±SD. Percentage (%) inhibition values were measured for drug-treated mice compared with vehicle-treated mice and are calculated as follows: Tumor growth inhibition (TGI, %)=100−[MTV treated/MTV control]×100. Significant differences between the treated versus the control groups (p<0.05) were determined using t test. At study endpoint, after blood collection, mice were practiced euthanasia by cervical dislocation, the tumor tissue was collected first, then abdominal cavity was cut open, liver and spleen were excised, then weight after the gallblader was removed respectively. Organ weight and Organ/body weight ratios between the treated versus the control groups were compared. Ratios was calculated as follows: Ratios=Organ weight/(body weight-tumor weight). Both organ weight and Organ/body weight ratios were also expressed as mean±SD, and significant differences between the treated versus the control groups (p<0.05) were determined using t test.

The following TABLE B lists compounds representative of the invention and their activity in subcutaneous EML4-ALK-BAF3 cell line xenograft model in nude mice described above. Compound 18 and Crizotinib were dosed at 40 mg/kg by oral gavage once daily for number of days. Tumor growth inhibition (TGI, %) was calculated. Compound 18 showed significant better tumor growth inhibition compared with Crizotinib. At the end of the study, the liver weight was measured and it was 1.318 gram for compound 18, 1.172 grams for Crizotinib and 1.523 for the control group respectively.

TABLE B
Tumor growth inhibition (TGI, %)

| | Compound | |
|---|---|---|
| Days | Crizotinib | Compound 18 |
| 4 | 20.85% | 24.11% |
| 7 | 30.43% | 38.91% |
| 9 | 27.98% | 39.08% |

The following TABLE C lists compounds representative of the invention and their activity in subcutaneous EML4-ALK-BAF3 cell line xenograft model in nude mice described above. Compound 20 and 22 were dosed at 40 mg/kg by oral gavage needle once daily for number of days. Tumor growth inhibition (TGI, %) was calculated. Compound 20 and 22 show significant tumor growth inhibition.

TABLE C
Tumor growth inhibition (TGI, %)

| | Compound | |
|---|---|---|
| Days | 20 | 22 |
| 2 | 29.80% | 22.58% |
| 5 | 58.20% | 53.72% |
| 8 | 71.73% | 66.61% |
| 11 | 90.29% | 82.97% |
| 14 | 92.09% | 87.64% |

Synthesis of Compounds

In synthesizing a compound of formulas I according to a desired procedure, the steps in some embodiment, are performed in an order suitable to prepare the compound, including a procedure described herein or by an alternate order of steps described herein, and in one embodiment, be preceded, or followed, by additional protection/deprotection steps as necessary. The intermediates in some embodiments are isolated or carried on in situ, with or without purification. Synthetic chemistry transformations and protecting group methodologies (protection and deprotection) useful in synthesizing the inhibitor compounds described herein are known in the art and include, for example, those such as described in R. Larock, Comprehensive Organic Transformations, VCH Publishers (1989); T. W. Greene and P. G. M. Wuts, Protective Groups in Organic Synthesis, 3^{rd} edition, John Wiley and Sons (1999); L. Fieser and M. Fieser, Fieser and Fieser's Reagents for Organic Synthesis, John Wiley and Sons (1994); A. Katritzky and A. Pozharski, Handbook of Heterocyclic Chemistry, 2^{nd} edition (2001); M. Bodanszky, A. Bodanszky, The Practice of Peptide Synthesis, Springer-Verlag, Berlin Heidelberg (1984); J. Seyden-Penne, Reductions by the Alumino- and Borohydrides in Organic Synthesis, 2^{nd} edition, Wiley-VCH, (1997); and L. Paquette, editor, Encyclopedia of Reagents for Organic Synthesis, John Wiley and Sons (1995).

Starting materials of the invention, are either known, commercially available, or can be synthesized in analogy to or according to methods that are known in the art. Many starting materials may be prepared according to known processes and, in particular, can be prepared using processes described in the examples. In synthesizing starting materials, functional groups in some cases are protected with suitable protecting groups when necessary. Protecting groups, their introduction and removal are described above.

The compounds of Formulas I were synthesized according to the procedures described in the following Schemes, wherein the substituents are as defined for Formulas I above, except where further noted. The synthetic methods described below are merely exemplary, and the compounds of the invention may also be synthesized by alternate routes.

The synthesis of compounds in the invention was described in the Scheme 1.

The amino group of A (“P” represents a protecting group of nitrogen in piperidine, such as Boc, Fmoc, or Cbz, etc.) can be substituted by acylation or alkylation to generate B. The protecting group of B was deprotected to generate C. C could be further alkylated to afford D.

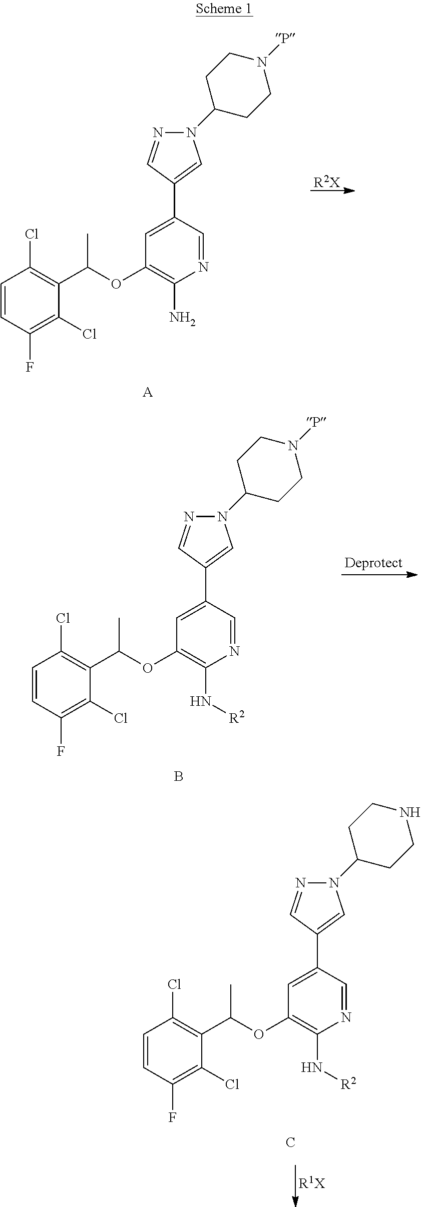

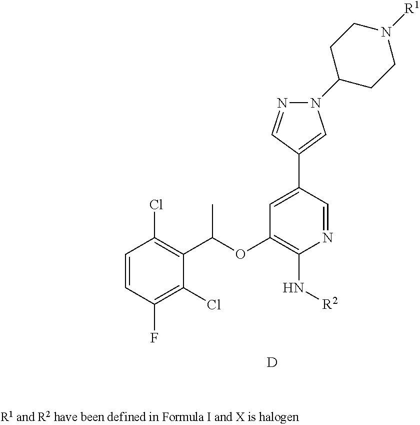

The synthesis of compounds 1 to 9 were described in Scheme 2.

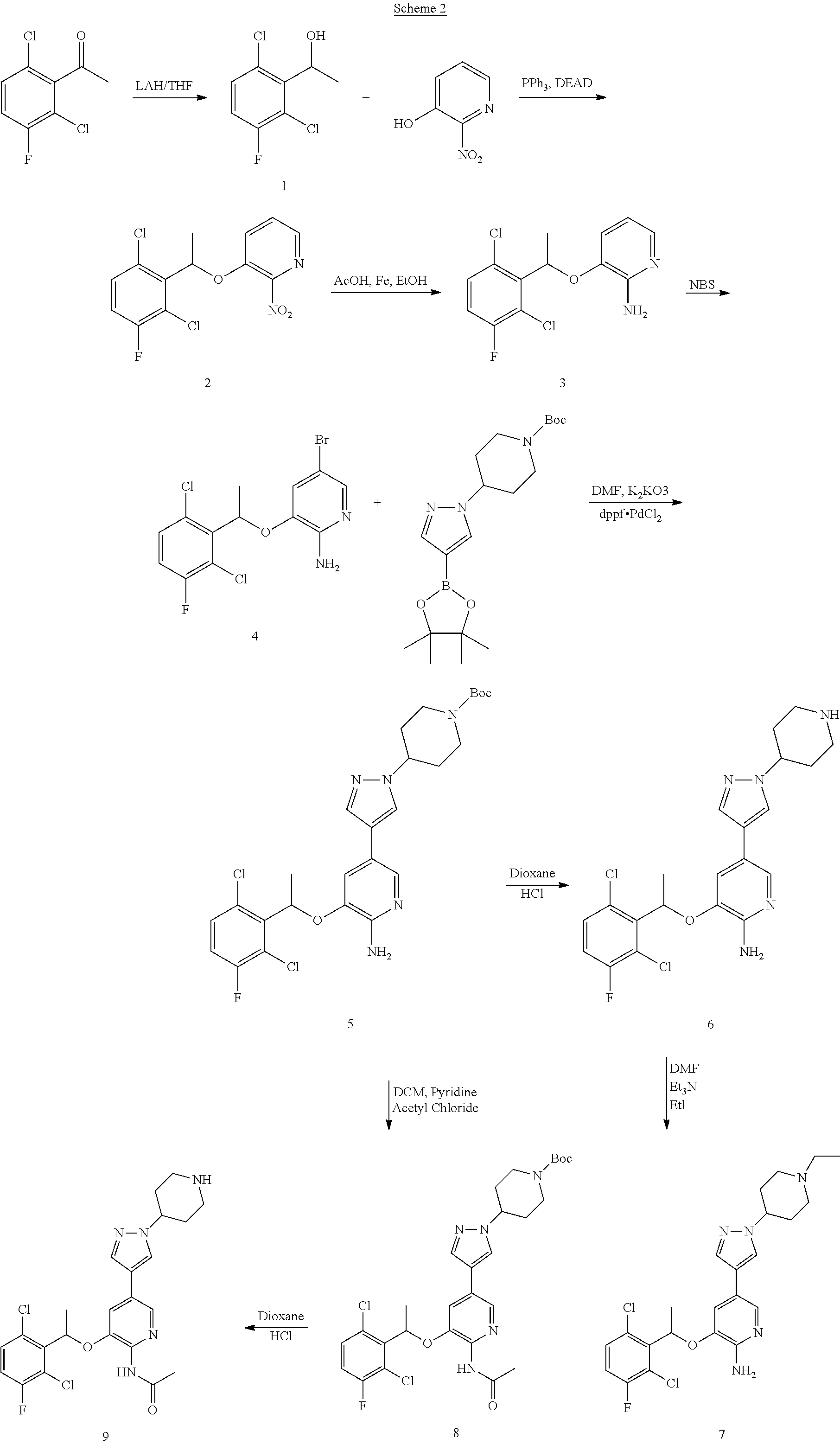

### DESCRIPTION OF EMBODIMENTS

### Proton NMR Spectra

Unless otherwise indicated, all ¹H NMR spectra were run on a Varian series Mercury 300, 400, 500 MHz instrument or a Bruker series 400, 500 MHz instrument. Where so characterized, all observed protons are reported as parts-per-million (ppm) downfield from tetramethylsilane (TMS) or other internal reference in the appropriate solvent indicated.

### Example 1

### Synthesis of 3-(1-(2,6-dichloro-3-fluorophenyl)ethoxy)-5-(1-(1-ethylpiperidin-4-yl)-1H-pyrazol-4-yl)pyridin-2-amine (Compound 7)

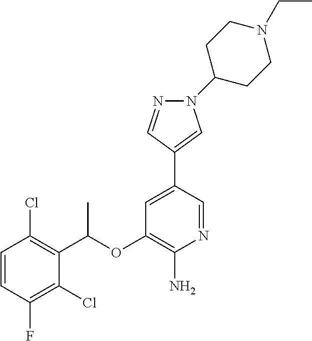 [SMILES: CCN1CCC(n2cc(-c3cnc(N)c(OC(C)c4c(Cl)ccc(F)c4Cl)c3)cn2)CC1]

### Step 1. 1-(2,6-Dichloro-3-fluoro-phenyl)ethanol (Compound 1)

2,6-Dichloro-3-fluoroacetophenone (3 g, 14.5 mmol, 1.0 eq) was stirred in THF at 0° C. using ice bath for 10 minutes and lithium aluminum hydride (551 mg, 14.5 mmol, 1.0 eq) was slowly added. The reaction was stirred at rt for 3 hours. The reaction was cooled in ice bath, and water was added dropwisely followed by adding 15% NaOH (0.6 mL) slowly. The mixture was stirred at rt for 30 min, 15% NaOH (2 mL), MgSO₄ were added and the mixture filtered to remove solids. The solids were washed with THF and the filtrate was concentrated to give 1-(2,6-Dichloro-3-fluoro-phenyl)ethanol (2.8 g) as a yellow oil. Yield (92.3%).

### Step 2. 3-(1-(2,6-dichloro-3-fluorophenyl)ethoxy)-2-nitropyridine (Compound 2)

To a stirred solution of triphenyl phosphine (2.9 g, 11.1 mmol, 1.5 eq) and DEAD (1.8 g, 10.4 mmol, 1.4 eq) in THF at 0° C. was added a solution of 1-(2,6-Dichloro-3-fluoro-phenyl)ethanol (1.55 g, 7.4 mmol, 1.0 eq) and 3-hydroxy-2-nitropyridine (1.14 g, 8.1 mmol, 1.1 eq). The resulting bright orange solution was stirred under a nitrogen at rt for 4 h at which point all starting materials had been consumed. The solvent was removed, and the crude was purified by chromatography on silica gel to give 3-(1-(2,6-dichloro-3-fluorophenyl)ethoxy)-2-nitropyridine (1.2 g) as a pink solid. Yield (48.6%).

### Step 3. 3-(1-(2,6-dichloro-3-fluorophenyl)ethoxy)pyridin-2-amine (Compound 3)

To a solution of AcOH (50 mL) and EtOH (30 mL) was added 3-(1-(2,6-dichloro-3-fluorophenyl)ethoxy)-2-nitropyridine (1.1 g, 3.3 mmol, 1.0 eq) and iron chips (1.9 g, 33.2 mmol, 10 eq). The reaction was heated slowly to reflux and allowed to stir for 1 h. The reaction was cooled to rt and then ethyl acetate and water was added. The solution was carefully neutralized by the addition of sodium carbonate. The combined organic extracts were washed with saturated bicarbonate sodium, water and brine, then dried over anhydrous Na₂SO⁴, filtered and concentrated to dryness under vacuum to yield 3-(1-(2,6-dichloro-3-fluorophenyl)ethoxy)pyridin-2-amine (950 mg) as a light pink solid. Yield (86.3%).

### Step 4. 5-bromo-3-(1-(2,6-dichloro-3-fluorophenyl)ethoxy)pyridin-2-amine (Compound 4)

A stirring solution 3-(1-(2,6-dichloro-3-fluorophenyl)ethoxy)pyridin-2-amine (1.0 g, 3.0 mmol, 1.0 eq) in acetonitrile was cooled to 0° C. using an ice bath. To the solution was added N-bromosuccinimide (546.6 mg, 3.2 mmol, 1.05 eq) portion wise. The reaction was stirred at 0° C. for 15 min. The reaction was concentrated to dryness under vacuum. The resulting dark oil was dissolved in ethyl acetate, and purified by column chromatography on silica gel to give 5-bromo-3-(1-(2,6-dichloro-3-fluorophenyl)ethoxy)pyridin-2-amine (850 mg) as a white solid. Yield (68.7%).

### Step 5. tert-butyl 4-(4-(6-amino-5-(1-(2,6-dichloro-3-fluorophenyl)ethoxy) pyridin-3-yl)-1H-pyrazol-1-yl)piperidine-1-carboxylate (Compound 5)

A mixture of tert-butyl 4-(4,5-dihydro-4-(4,4,5,5-tetramethyl-1,3,2-dioxaborolan-2-yl) pyrazol-1-yl)piperidine-1-carboxylate (760 mg, 2.0 mmol, 1.0 eq), 5-bromo-3-(1-(2,6-dichloro-3-fluorophenyl)ethoxy)pyridin-2-amine (943 mg, 2.5 mmol, 1.25 eq), and Na₂CO₃ (636 mg, 6.0 mmol, 3.0 eq) under a nitrogen atmosphere was treated with DMF (20 mL), water (5 mL), and Pd(dppf)Cl₂(73 mg, 0.1 mmol, 0.05 eq). The mixture was purged with bubbling nitrogen for 2 minutes, and then stirred at 100° C. overnight, cooled to room temperature, poured into water (100 mL), and extracted with ethyl acetate (6×50 mL). The combined organic layers were washed with brine, dried (MgSO₄), filtered, and concentrated. The crude product was purified by flash column chromatography on silica gel to give the title compound (800 mg). Yield (72.7%).

### Step 6. 3-(1-(2,6-dichloro-3-fluorophenyl)ethoxy)-5-(1-(piperidin-4-yl)-1H-pyrazol-4-yl)pyridin-2-amine (Compound 6)

To a solution of tert-butyl 4-(4-(6-amino-5-(1-(2,6-dichloro-3-fluorophenyl) ethoxy)pyridin-3-yl)-1H-pyrazol-1-yl)piperidine-1-carboxylate (330 mg, 0.6 mmol, 1.0 eq) in DCM (20 mL) was added saturated dioxane of HCl (7 mL). The reaction mixture was stirred at rt overnight until TLC indicated the consumption of starting material. The pH of the reaction mixture was adjusted to 8 by saturated bicarbonate sodium. The aqueous was extracted with ethyl acetate (8×20 mL), the combined organic layers were washed with brine, dried (MgSO₄), filtered, and concentrated. The crude product was purified by column chromatography on silica gel to give the title compound 240 mg (89% yield).

### Step 7. 3-(1-(2,6-dichloro-3-fluorophenyl)ethoxy)-5-(1-(1-ethylpiperidin-4-yl)-1H-pyrazol-4-yl)pyridin-2-amine

To a solution of 3-(1-(2,6-dichloro-3-fluorophenyl)ethoxy)-5-(1-(piperidin-4-yl)-1H-pyrazol-4-yl)pyridin-2-amine (45 mg, 0.1 mmol) and Et₃N (30 mg, 0.3 mmol, 3.0 eq) in DMF (2 mL) was added EtI (19 mg, 0.12 mmol, 1.2 eq). The reaction mixture was stirred at rt overnight. TLC indicated the consumption of starting material and quenched with water (20 mL). The aqueous was extracted with ethyl acetate (8×20 mL), the combined organic layers were washed with brine, dried (MgSO₄), filtered, and concentrated. The crude product was purified by column chromatography on silica gel to give 3-(1-(2,6-dichloro-3-fluorophenyl) ethoxy)-5-(1-(1-ethylpiperidin-4-yl)-1H-pyrazol-4-yl)pyridin-2-amine as an off-white solid 35 mg (73.2% yield). H-NMR-PH-NC-LX-007-0 (400 MHz, CDCl3, ppm): 7.76-7.57 (1H, d), 7.56 (1H, s), 7.50 (1H, s), 7.33-7.28 (1H, m), 7.05 (1H, t, J=11.72 Hz), 6.87 (1H, m), 6.11.-6.04 (1H, dd), 4.75 (2H, s), 4.14 (1H, m), 3.17-3.01 (1H, m), 2.50-2.45 (2H, m), 2.17-2.02 (6H, m), 1.86-1.84 (3H, d, J=8.8 Hz), 1.16-1.11 (3H, t, J=19.2 Hz).

### Example 2

### Synthesis of N-(3-(1-(2,6-dichloro-3-fluorophenyl)ethoxy)-5-(1-(piperidin-4-yl)-1H-pyrazol-4-yl)pyridin-2-yl)acetamide (Compound 9)

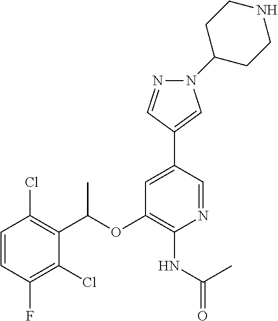 [SMILES: CC(=O)Nc1ncc(-c2cnn(C3CCNCC3)c2)cc1OC(C)c1c(Cl)ccc(F)c1Cl]

### Step 1. tert-butyl 4-(4-(6-acetamido-5-(1-(2,6-dichloro-3-fluorophenyl) ethoxy)pyridin-3-yl)-1H-pyrazol-1-yl)piperidine-1-carboxylate (Compound 8)

To a solution of tert-butyl 4-(4-(6-amino-5-(1-(2,6-dichloro-3-fluorophenyl) ethoxy)pyridin-3-yl)-1H-pyrazol-1-yl)piperidine-1-carboxylate (500 mg, 0.91 mmol) and pyridine (288 mg, 3.64 mmol, 4.0 eq) in DCM (20 mL) was added acetyl chloride (87 mg, 1.1 mmol, 1.2 eq). The reaction mixture was stirred at room temperature overnight. TLC indicated the consumption of starting material and the reaction was quenched with water (20 mL). The aqueous was extracted with ethyl acetate (8×20 mL), the combined organic layers were washed with brine, dried (MgSO₄), filtered, and concentrated. The crude product was purified by column chromatography on silica gel to give tert-butyl 4-(4-(6-acetamido-5-(1-(2,6-dichloro-3-fluorophenyl)ethoxy)pyridin-3-yl)-1H-pyrazol-1-yl) piperidine-1-carboxylate as an off-white solid 450 mg (83.5% yield).

### Step 2. N-(3-(1-(2,6-dichloro-3-fluorophenyl)ethoxy)-5-(1-(piperidin-4-yl)-1H-pyrazol-4-yl)pyridin-2-yl)acetamide

To a solution of tert-butyl 4-(4-(6-acetamido-5-(1-(2,6-dichloro-3-fluorophenyl)ethoxy)pyridin-3-yl)-1H-pyrazol-1-yl)piperidine-1-carboxylate (380 mg, 0.68 mmol, 1.0 eq) in ethyl acetate (15 mL) was added saturated dioxane of HCl (15 mL). The reaction mixture was stirred at room temperature overnight until TLC indicated the consumption of starting material. The pH of the reaction mixture was adjusted to 8 by saturated bicarbonate sodium. The aqueous was extracted with ethyl acetate (8×20 mL), the combined organic layers were washed with brine, dried (MgSO4), filtered, and concentrated. The crude product was purified by column chromatography on silica gel to give N-(3-(1-(2,6-dichloro-3-fluorophenyl)ethoxy)-5-(1-(piperidin-4-yl)-1H-pyrazol-4-yl)pyridin-2-yl) acetamide as off-white solid 197 mg. (58.8% yield). H-NMR-PH-NC-LX-007-0 (400 MHz, DMSO-d6, ppm): 8.07 (1H, s), 8.00 (1H, s, br), 7.56 (1H, s), 7.50 (1H, s), 7.27-7.22 (1H, m), 7.23 (1H, s), 6.07.-6.03 (1H, m), 4.20-4.14 (1H, m), 3.22-3.19 (2H, d, J=12.8 Hz), 2.73 (2H, t, J=12 Hz), 2.13-2.10 (2H, m), 1.90-1.77 (8H, m, J), 1.19-1.17 (3H, m).

### Example 3

### Synthesis of N-(3-(1-(2,6-dichloro-3-fluorophenyl)ethoxy)-5-(1-(1-ethylpiperidin-4-yl)-1H-pyrazol-4-yl)pyridin-2-yl)acetamide (Compound 10)

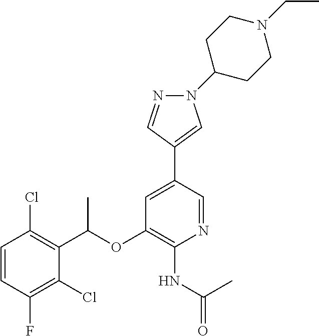 [SMILES: CCN1CCC(n2cc(-c3cnc(NC(C)=O)c(OC(C)c4c(Cl)ccc(F)c4Cl)c3)cn2)CC1]

To a solution of N-(3-(1-(2,6-dichloro-3-fluorophenyl)ethoxy)-5-(1-(piperidin-4-yl)-1H-pyrazol-4-yl)pyridin-2-yl)acetamide (45 mg, 0.09 mmol, 1.0 eq) and Et₃N (28 mg, 0.18 mmol, 3.0 eq) in DMF (2 mL) in an ice bath was added bromoethane (20 mg, 0.18 mmol, 2.0 eq) slowly in portions. The reaction mixture was stirred at room temperature for 20 hours. TLC indicated the consumption of starting material and quenched with water (20 mL), extracted with ethyl acetate (3×20 mL), the combined organic layers were washed with brine, dried (MgSO₄), filtered, and concentrated. The crude product was purified by silica gel column to give N-(3-(1-(2,6-dichloro-3-fluorophenyl)ethoxy)-5-(1-(1-ethylpiperidin-4-yl)-1H-pyrazol-4-yl)pyridin-2-yl)acetamide 20 mg. ¹H-NMR (DMSO-d⁶, 500 Hz): δ1.12-1.18 (m, 3H), δ1.78 (d, J=6.5 Hz, 3H), δ2.06 (s, 3H), δ2.10-2.14 (m, 2H), δ2.70-2.81 (m, 2H), δ3.09-3.1 (m, 2H), δ3.36-3.42 (m, 2H), δ4.25-4.33 (m, 1H), δ5.60-5.75 (m, 1H), δ6.13-6.17 (m, 1H), δ7.36 (s, 1H), δ7.15-7.19 (m, 1H), δ7.49-7.54 (m, 2H), δ7.79 (s, 1H), δ8.22 (s, 1H), δ9.45 (s, 1H).

### Example 4

### Synthesis of 3-(1-(2,6-dichloro-3-fluorophenyl)ethoxy)-N-ethyl-5-(1-(piperidin-4-yl)-1H-pyrazol-4-yl)pyridin-2-amine (Compound 11)

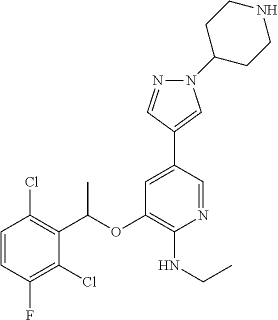 [SMILES: CCNc1ncc(-c2cnn(C3CCNCC3)c2)cc1OC(C)c1c(Cl)ccc(F)c1Cl]

Step 1. To a solution of tert-butyl 4-(4-(5-(1-(2,6-dichloro-3-fluorophenyl) ethoxy)-6-aminopyridin-3-yl)-1H-pyrazol-1-yl)piperidine-1-carboxylate (150 mg, 0.27 mmol, 1.0 eq) in DMF (2 mL) was added NaH (29 mg, 1.2 mmol, 4.4 eq) in an ice bath. The reaction mixture was stirred at rt for 0.5 hour. Then iodoethane (54 mg, 0.35 mmol, 1.27 eq) in DMF (1 mL) was added, and stirred for overnight at rt. The resulting mixture was quenched with 20 ml H₂O, extracted with EA (3×20 mL). The crude product was purified by silica gel column to give tert-butyl 4-(4-(5-(1-(2,6-dichloro-3-fluorophenyl)ethoxy)-6-(ethylamino)pyridin-3-yl)-1H-pyrazol-1-yl)piperidine-1-carboxylate as an off-white solid 85 mg.

Step 2. To a solution of tert-butyl 4-(4-(5-(1-(2,6-dichloro-3-fluorophenyl) ethoxy)-6-(ethylamino)pyridin-3-yl)-1H-pyrazol-1-yl)piperidine-1-carboxylate (85 mg, 0.147 mmol, 1.0 eq) in THF (1 mL) was added concentrated HCl (1 mL) in an ice bath. The reaction mixture was stirred at rt for 2 hours. The pH of the reaction mixture was adjusted to 9 by saturated bicarbonate sodium. The aqueous solution was extracted with ethyl acetate (3×20 mL), the combined organic layers were washed with brine, dried (MgSO4), filtered, and concentrated. The crude product was purified by prep-TLC to give the title compound 21 mg.

¹H-NMR (DMSO-d⁶, 500 Hz): δ1.57 (d, J=6.2 Hz, 3H), δ1.72-2.06 (m, 6H), δ2.10 (m, 3H), δ2.35-2.59 m, 2H), δ3.01-3.04 (m, 2H), δ4.15-4.20 (m, 1H), δ5.79-5.82 (m, 1H), δ7.16-7.20 (m, 1H), δ7.23 (s, 1H), δ7.52-7.55 (m, 1H), δ7.64-7.66 (m, 1H), δ7.74 (s, 1H), δ8.18 (s, 1H), δ8.19 (s, 1H). δ9.73 (s, 1H).

### Example 5

### Synthesis of 3-(1-(2,6-dichloro-3-fluorophenyl)ethoxy)-5-(1-(piperidin-4-yl)-1H-pyrazol-4-yl)-N-propylpyridin-2-amine (Compound 12)

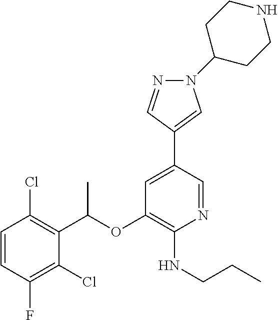 [SMILES: CCCNc1ncc(-c2cnn(C3CCNCC3)c2)cc1OC(C)c1c(Cl)ccc(F)c1Cl]

Compound 12 was prepared as an off-white solid from tert-butyl 4-(4-(5-(1-(2,6-dichloro-3-fluorophenyl)ethoxy)-6-aminopyridin-3-yl)-1H-pyrazol-1-yl)piperidine-1-carboxylate and iodo propane as an off-white solid using a similar procedure that described for the synthesis of compound 11. ¹H-NMR (DMSO-d⁶, 500 Hz): δ0.88-0.91 (m, 3H), δ1.23 (m, 2H), δ1.55-1.62 (m, 5H), δ1.80-2.00 (m, 4H), δ2.68-2.73 (m, 2H), δ3.11-3.14 (m, 2H), δ4.22 (m, 1H), δ5.82-5.83 (m, 1H), δ6.15-6.17 (m, 1H), δ6.98 (s, 1H), δ7.15-7.19 (m, 1H), δ7.49-7.54 (m, 2H), δ7.59 (s, 1H), δ7.79 (s, 1H), δ7.91 (s, 1H).

### Example 6

### Synthesis of (S)—N-(3-(1-(2,6-dichloro-3-fluorophenyl)ethoxy)-5-(1-(piperidin-4-yl)-1H-pyrazol-4-yl)pyridin-2-yl)acetamide (Compound 13)

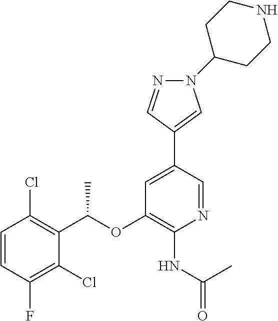 [SMILES: CC(=O)Nc1ncc(-c2cnn(C3CCNCC3)c2)cc1O[C@@H](C)c1c(Cl)ccc(F)c1Cl]

Compound 13 was prepared as an off-white solid from (S)-tert-butyl 4-(4-(6-amino-5-(1-(2,6-dichloro-3-fluorophenyl)ethoxy)pyridin-3-yl)-1H-pyrazol-1-yl)piperidine-1-carboxylate (using a similar procedure that described for the synthesis of Compound tert-butyl 4-(4-(6-amino-5-(1-(2,6-dichloro-3-fluorophenyl)ethoxy)pyridin-3-yl)-1H-pyrazol-1-yl)piperidine-1-carboxylate from (R)-1-(2,6-dichloro-3-fluorophenyl)ethanol) and acetyl chloride as an off-white solid using a similar procedure that described for the synthesis of compound 9. ¹H-NMR (DMSO-d⁶, 300 Hz): δ1.80 (d, J=6.3 Hz, 3H), δ2.10 (s, 3H), δ2.20-2.22 (m, 2H), δ3.06-3.10 (m, 2H), δ3.39-3.41 (m, 2H), δ3.57-3.60 (m, 2H), δ4.5-4.6 (m, 1H), δ5.75-5.80 (m, 1H), δ6.15-6.25 (m, 1H), δ7.42-7.48 (m, 1H), δ7.54-7.95 (m, 1H), δ7.84 (s, 1H), δ8.23 (s, 2H), δ8.93 (brs, 1H), 9.48 (brs, 1H).

### Example 7

### Synthesis of (S)-3-(1-(2,6-dichloro-3-fluorophenyl)ethoxy)-5-(1-(1-methylpiperidin-4-yl)-1H-pyrazol-4-yl)pyridin-2-amine (Compound 14)

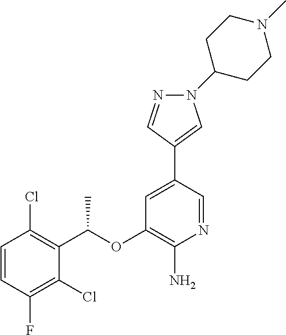 [SMILES: C[C@H](Oc1cc(-c2cnn(C3CCN(C)CC3)c2)cnc1N)c1c(Cl)ccc(F)c1Cl]

Compound 14 was prepared as an off-white solid from (S)-3-(1-(2,6-dichloro-3-fluorophenyl)ethoxy)-5-(1-(piperidin-4-yl)-1H-pyrazol-4-yl)pyridin-2-am ine and iodo methane as an off-white solid using a similar procedure that described for the synthesis of compound 7. ¹H-NMR (DMSO-d⁶, 500 Hz): δ1.80 (d, J=6.6 Hz, 3H), δ1.92-1.96 (m, 4H), δ2.08 (m, 2H), δ2.22 (s, 3H), δ2.85-2.87 (m, 2H), δ4.05-4.10 (m, 1H), δ5.61 (s, 2H), δ6.06-6.10 (m, 1H), δ6.89 (s, 1H), δ7.41-7.45 (m, 1H), δ7.52 (s, 1H), δ7.55-7.58 (m, 1H), δ7.75 (s, 1H), δ7.92 (s, 1H).

### Example 8

### Synthesis of (S)-3-(1-(2,6-dichloro-3-fluorophenyl)ethoxy)-5-(1-(1-d₃-methyl-piperidin-4-yl)-1H-pyrazol-4-yl)pyridin-2-amine (Compound 15)

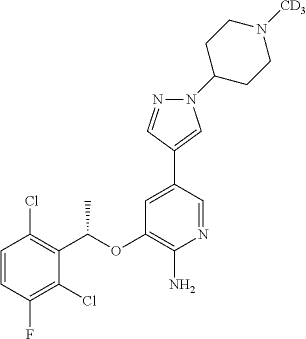 [SMILES: [2H]C([2H])([2H])N1CCC(n2cc(-c3cnc(N)c(O[C@@H](C)c4c(Cl)ccc(F)c4Cl)c3)cn2)CC1]

Compound 15 was prepared as an off-white solid from (S)-3-(1-(2,6-dichloro-3-fluorophenyl)ethoxy)-5-(1-(piperidin-4-yl)-1H-pyrazol-4-yl)pyridin-2-am ine and CD₃I as an off-white solid using a similar procedure that described for compound 7. ¹H-NMR (DMSO-d⁶, 500 Hz): δ1.79 (d, J=6.6 Hz, 3H), δ1.91-1.98 (m, 4H), δ2.02-2.07 (m, 2H), δ2.83-2.85 (m, 2H), δ4.03-4.09 (m, 1H), δ5.61 (s, 2H), δ6.06-6.10 (m, 1H), δ6.88 (s, 1H), δ7.41-7.45 (m, 1H), δ7.52 (s, 1H), δ7.55-7.58 (m, 1H), δ7.75 (s, 1H), δ7.79 (s, 1H).

### Example 9

### Synthesis of (S)-3-(1-(2,6-dichloro-3-fluorophenyl)ethoxy)-5-(1-(1-ethylpiperidin-4-yl)-1H-pyrazol-4-yl)pyridin-2-amine (Compound 16)

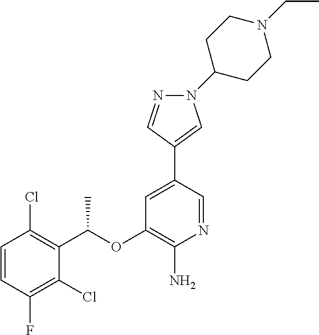 [SMILES: CCN1CCC(n2cc(-c3cnc(N)c(O[C@@H](C)c4c(Cl)ccc(F)c4Cl)c3)cn2)CC1]

Compound 16 was prepared as an off-white solid from (S)-3-(1-(2,6-dichloro-3-fluorophenyl)ethoxy)-5-(1-(piperidin-4-yl)-1H-pyrazol-4-yl)pyridin-2-am ine and iodoethane as an off-white solid using a similar procedure that described for the synthesis of compound 7. ¹H-NMR (DMSO-d⁶, 500 Hz): δ1.00-1.01 (m, 3H), δ1.79-1.80 (d, J=6.55 Hz, 3H), δ1.89-1.93 (m, 2H), δ1.97-2.03 (m, 4H), δ2.36 (m, 2H), δ2.93-2.95 (m, 2H), δ4.08 (m, 1H), δ5.60 (s, 2H), δ6.06-6.10 (m, 1H), δ6.89 (s, 1H), δ7.41-7.45 (m, 1H), δ7.52 (s, 1H), δ7.55-7.57 (m, 1H), δ7.74 (s, 1H), δ7.93 (s, 1H).

### Example 10

### Synthesis of (S)-3-(1-(2,6-dichloro-3-fluorophenyl)ethoxy)-5-(1-(1-d₅-ethyl-piperidin-4-yl)-1H-pyrazol-4-yl)pyridin-2-amine (Compound 17)

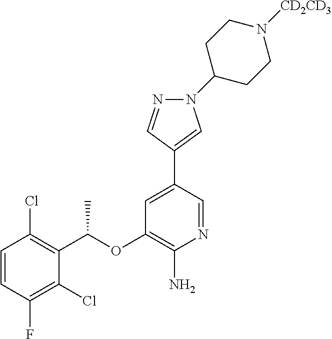

Compound 17 was prepared as an off-white solid from (S)-3-(1-(2,6-dichloro-3-fluorophenyl)ethoxy)-5-(1-(piperidin-4-yl)-1H-pyrazol-4-yl) pyridin-2-amine and CD₂CD₃I using a similar procedure that described for the synthesis of compound 7.

¹H-NMR (DMSO-d⁶, 500 Hz): δ1.80 (d, J=6.6 Hz, 3H), δ1.89-1.93 (m, 2H), δ1.97-2.02 (m, 4H), δ2.92-2.94 (m, 2H), δ4.07 (m, 1H), δ5.60 (s, 1H), δ6.06-6.10 (m, 1H), δ6.89 (s, 1H), δ7.41-7.45 (m, 1H), δ7.51 (s, 1H), δ7.55-7.57 (m, 1H), δ7.74 (s, 1H), δ7.93 (s, 1H).

### Example 11

### Synthesis of (R)—N-(3-(1-(2,6-dichloro-3-fluorophenyl)ethoxy)-5-(1-(piperidin-4-yl)-1H-pyrazol-4-yl)pyridin-2-yl)acetamide (Compound 18)

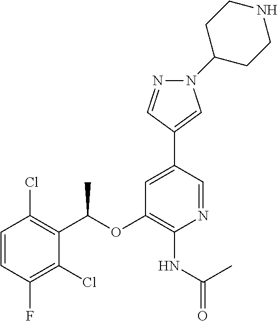 [SMILES: CC(=O)Nc1ncc(-c2cnn(C3CCNCC3)c2)cc1O[C@H](C)c1c(Cl)ccc(F)c1Cl]

Step 1. To a solution of (R)-tert-butyl 4-(4-(6-amino-5-(1-(2,6-dichloro-3-fluorophenyl)ethoxy)pyridin-3-yl)-1H-pyrazol-1-yl)piperidine-1-carboxylate (4 g, 7.27 mmol, 1.0 eq) and pyridine (2.3 g, 29.1 mmol, 4.0 eq) in 50 ml DCM was added acetyl chloride (0.86 g, 10.9 mmol, 1.5 eq) in an ice bath. The reaction mixture was stirred at room temperature for overnight. The resulting mixture was washed with H₂O (3×20 mL). The organic layer was dried and concentrated. The crude product was purified on silica gel column to give (R)-tert-butyl 4-(4-(6-acetamido-5-(1-(2,6-dichloro-3-fluorophenyl)ethoxy)pyridin-3-yl)-1H-pyrazol-1-yl)piperidine-1-carboxylate 1.66 g (38.6% yield).

Step 2. To a solution of (R)-tert-butyl 4-(4-(6-acetamido-5-(1-(2,6-dichloro-3-fluorophenyl)ethoxy)pyridin-3-yl)-1H-pyrazol-1-yl)piperidine-1-carboxylate (500 mg, 0.84 mmol, 1.0 eq) in DCM (5 mL) was added trifluoroacetic acid (2 ml) in an ice bath. The reaction mixture was stirred at room temperature for 2 hours. The pH of the reaction mixture was adjusted to 9 by saturated bicarbonate sodium in an ice bath. The aqueous solution was extracted with ethyl acetate (3×20 mL), the combined organic layers were washed with brine, dried over (MgSO₄), filtered, and concentrated. The crude product was purified by silica gel column to give (R)—N-(3-(1-(2,6-dichloro-3-fluorophenyl)ethoxy)-5-(1-(piperidin-4-yl)-1H-pyrazol-4-yl)pyridin-2-yl)acetamide 250 mg (60.2% yield).

¹H-NMR (CDCl3, 400 Hz): δ1.88 (d, J=6.4 Hz, 3H), δ1.90-1.94 (m, 2H), δ2.16-2.20 (m, 2H), δ2.48 (s, 3H), δ2.76-2.824 (m, 2H), δ3.25-3.28 (m, 2H), δ3.69-3.74 (m, 1H), δ4.22-4.26 (m, 1H), δ6.10-6.15 (m, 1H), δ7.05-7.07 (m, 1H), δ7.09 (s, 1H), δ7.30-7.33 (m, 1H), δ7.59 (s, 1H), δ7.62 (s, 1H), δ8.06 (s, 1H), δ8.12 (s, 1H). MS m/z 493 [M+1]

### Example 12

### Synthesis of (R)-3-(1-(2,6-dichloro-3-fluorophenyl)ethoxy)-5-(1-(1-methylpiperidin-4-yl)-1H-pyrazol-4-yl)pyridin-2-amine (Compound 19)

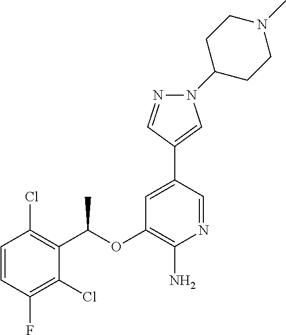 [SMILES: C[C@@H](Oc1cc(-c2cnn(C3CCN(C)CC3)c2)cnc1N)c1c(Cl)ccc(F)c1Cl]

Compound 19 was prepared as an off-white solid from (R)-3-(1-(2,6-dichloro-3-fluorophenyl)ethoxy)-5-(1-(piperidin-4-yl)-1H-pyrazol-4-yl)pyridin-2-am ine and iodo methane as an off-white solid using a similar procedure that described for the synthesis of compound 7. ¹H-NMR (DMSO-d⁶, 500 Hz): δ1.80 (d, J=6.6 Hz, 3H), δ1.94-1.99 (m, 4H), δ2.17-2.19 (m, 2H), δ2.28 (s, 3H), δ2.92 (m, 2H), δ4.11 (m, 1H), δ5.62 (s, 2H), δ6.06-6.10 (m, 1H), δ6.89 (s, 1H), δ7.41-7.45 (m, 1H), δ7.53 (s, 1H), δ7.55-7.57 (m, 1H), δ7.75 (s, 1H), δ7.93 (s, 1H). MS m/z 465 [M+1]

### Example 13

### Synthesis of (R)-3-(1-(2,6-dichloro-3-fluorophenyl)ethoxy)-5-(1-(1-d₃-methylpiperidin-4-yl)-1H-pyrazol-4-yl)pyridin-2-amine (compound 20)

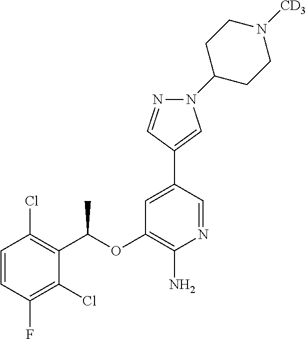 [SMILES: [2H]C([2H])([2H])N1CCC(n2cc(-c3cnc(N)c(O[C@H](C)c4c(Cl)ccc(F)c4Cl)c3)cn2)CC1]

Compound 20 was prepared as an off-white solid from (R)-3-(1-(2,6-dichloro-3-fluorophenyl)ethoxy)-5-(1-(piperidin-4-yl)-1H-pyrazol-4-yl)pyridin-2-am ine and CD₃I as an off-white solid using a similar procedure that described for the synthesis of compound 7. ¹H-NMR (DMSO-d⁶, 500 Hz): δ1.80 (d, J=6.6 Hz, 3H), δ1.91-1.98 (m, 4H), δ2.07 (m, 2H), δ2.84-2.87 (m, 2H), δ4.05-4.09 (m, 1H), δ5.62 (s, 2H), δ6.06-6.10 (m, 1H), δ6.89 (s, 1H), δ7.41-7.45 (m, 1H), δ7.52 (s, 1H), δ7.55-7.57 (m, 1H), δ7.75 (s, 1H), δ7.92 (s, 1H). MS m/z 468 [M+1]

### Example 14

### Synthesis of (R)-3-(1-(2,6-dichloro-3-fluorophenyl)ethoxy)-5-(1-(1-ethylpiperidin-4-yl)-1H-pyrazol-4-yl)pyridin-2-amine (Compound 21)

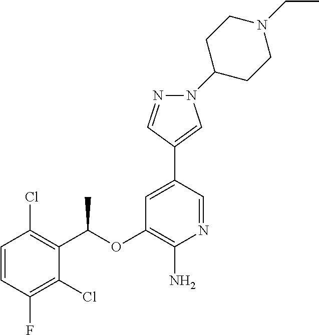 [SMILES: CCN1CCC(n2cc(-c3cnc(N)c(O[C@H](C)c4c(Cl)ccc(F)c4Cl)c3)cn2)CC1]

Compound 21 was prepared as an off-white solid from (R)-3-(1-(2,6-dichloro-3-fluorophenyl)ethoxy)-5-(1-(piperidin-4-yl)-1H-pyrazol-4-yl)pyridin-2-am ine and CH₂CH₃I as an off-white solid using a similar procedure that described for the synthesis of compound 7. ¹H-NMR (DMSO-d⁶, 500 Hz): δ1.01 (1, J=7.1 Hz, 3H), δ1.80 (d, J=7.4 Hz, 3H), δ1.89-2.02 (m, 6H), δ2.36 (m, 2H), δ2.93-2.95 (m, 2H), δ4.08 (m, 1H), δ5.62 (s, 2H), δ6.06-6.10 (m, 1H), δ6.89 (s, 1H), δ7.41-7.45 (m, 1H), δ7.51 (s, 1H), δ7.55-7.57 (m, 1H), δ7.74 (s, 1H), δ7.93 (s, 1H). MS m/z 478 [M+1]

### Example 15

### Synthesis of (R)-3-(1-(2,6-dichloro-3-fluorophenyl)ethoxy)-5-(1-(1-d₅-ethylpiperidin-4-yl)-1H-pyrazol-4-yl)pyridin-2-amine (Compound 22)

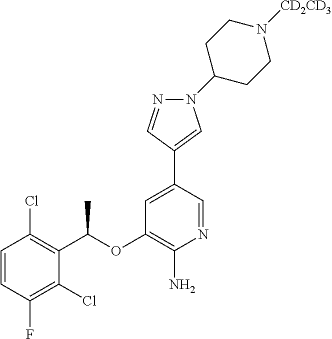

Compound 22 was prepared as an off-white solid from (R)-3-(1-(2,6-dichloro-3-fluorophenyl)ethoxy)-5-(1-(piperidin-4-yl)-1H-pyrazol-4-yl)pyridin-2-amine and CD₂CD₃I as an off-white solid using a similar procedure that described for the synthesis of compound 7. ¹H-NMR (DMSO-d⁶, 500 Hz): δ1.80 (d, J=6.6 Hz, 3H), δ1.89-2.02 (m, 6H), δ2.93 (m, 2H), δ4.08 (m, 1H), δ5.62 (s, 2H), δ6.06-6.10 (m, 1H), δ6.89 (s, 1H), δ7.41-7.45 (m, 1H), δ7.52 (s, 1H), δ7.55-7.57 (m, 1H), δ7.74 (s, 1H), δ7.93 (s, 1H). MS m/z 484 [M+1]

### Example 16

A representative protocol for the PK (pharmacokinetic) is as followed: Animals used were Wistar rat, male and weight about 300-359 grams. Compound 21 and 22 were given either IV or oral to the same animals by cassette dosing. The dose for each compound is 1 mg/kg (volume 5 ml/kg) IV and 2 mg/kg (volume 10 ml/kg) oral. The formulation for IV was in PBS, the pH is adjusted by dilute HCl until compounds were totally dissolved. 0.5% Na-CMC suspension was used for the oral formulation. Sample collection: for IV route, plasma sample were collected at the time point 0.083, 0.25, 0.5, 1, 1.5, 2, 4, 6, 8, 12, 24 h. For oral route, plasma sample were collected at the time point 0.167, 0.33, 0.5, 1, 2, 4, 6, 8, 12, 24 h. Analysis: LC-MS/MS is used to calculate PK parameters: t1/2, tmax, Cmax, Vss and AUC etc.

The following TABLE D lists compounds 21 and 22 and their PK properties by IV cassette 1 mg/kg each dosing to the same animals.

TABLE D

| | | Cmax | AUC0-t | AUC0-∞ | | | Cl |
|---|---|---|---|---|---|---|---|
| | Tmax h | ng/ml | ng · h/mL | ng · h/mL | MRT h | T_{1/2 h} | mL/h/kg |
| 21 | 0.083 | 317 | 442.35 | 443.23 | 2.89 | 2.89 | 2262 |
| 22 | 0.083 | 259 | 336.42 | 337.34 | 3.11 | 3.04 | 2973 |

The following TABLE E lists compounds 21 and 22 and their PK properties by oral cassette 2 mg/kg dosing each to the same animals.

TABLE E

| | | Cmax | AUC0-t | AUC0-∞ | | | |
|---|---|---|---|---|---|---|---|
| | Tmax h | ng/ml | ng · h/mL | ng · h/mL | MRT h | T_{1/2 h} | F % |
| 21 | 4 | 22.9 | 148.34 | 154.01 | 4.97 | 2.99 | 16.82 |
| 22 | 4 | 21 | 139.68 | 146.46 | 5.28 | 3.1 | 20.77 |

Compound 22 had bioavailability of 20.77% and compound 21 had bioavailability of 16.82%.

1-13. (canceled)

14. A compound of Formula I: 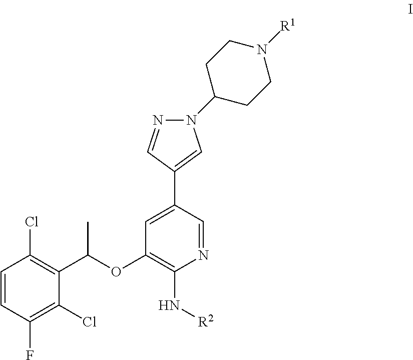
or a pharmaceutically acceptable salt, solvate, prodrug or enantiomer thereof, wherein
R¹ is hydrogen, substituted or unsubstituted C₁₋₆ alkyl;
R² is hydrogen, substituted or unsubstituted C₁₋₆ alkyl, —C(O)R³ or —C(O)OR³;
R³ is substituted or unsubstituted C₁₋₆ alkyl;
with the proviso that R¹ and R² are not both hydrogen, and R¹ is substituted or unsubstituted deuterated C₁₋₆ alkyl when R² is hydrogen.

15. The compound of claim 14, wherein R¹ is selected from the group consisting of hydrogen, methyl, and ethyl.

16. The compound of claim 14, wherein R¹ is —CD₃ or —CD₂CD₃.

17. The compound of claim 14, wherein R² is selected from the group consisting of hydrogen, —C(O)CH₃ (Acetyl), —C(O)CH₂CH₃, and —C(O)OCH₂CH₃.

18. A compound or its pharmaceutically acceptable salt, or solvate thereof selected from the group consisting of:
3-(1-(2,6-dichloro-3-fluorophenyl)ethoxy)-5-(1-(1-ethylpiperidin-4-yl)-1H-pyrazol-4-yl)pyridin-2-amine;
N-(3-(1-(2,6-dichloro-3-fluorophenyl)ethoxy)-5-(1-(piperidin-4-yl)-1H-pyrazol-4-yl)pyridin-2-yl)acetamide;
N-(3-(1-(2,6-dichloro-3-fluorophenyl)ethoxy)-5-(1-(1-methylpiperidin-4-yl)-1H-pyrazol-4-yl)pyridin-2-yl)acetamide;
N-(3-(1-(2,6-dichloro-3-fluorophenyl)ethoxy)-5-(1-(1-ethylpiperidin-4-yl)-1H-pyrazol-4-yl)pyridin-2-yl)acetamide;
3-(1-(2,6-dichloro-3-fluorophenyl)ethoxy)-N-ethyl-5-(1-(piperidin-4-yl)-1H-pyrazol-4-yl)pyridin-2-amine;
3-(1-(2,6-dichloro-3-fluorophenyl)ethoxy)-5-(1-(piperidin-4-yl)-1H-pyrazol-4-yl)-N-propylpyridin-2-amine;
3-(1-(2,6-dichloro-3-fluorophenyl)ethoxy)-5-(1-(1-d₃-methyl-piperidin-4-yl)-1H-pyrazol-4-yl)pyridin-2-amine;
3-(1-(2,6-dichloro-3-fluorophenyl)ethoxy)-5-(1-(1-d₅-ethyl-piperidin-4-yl)-1H-pyrazol-4-yl)pyridin-2-amine;
(S)—N-(3-(1-(2,6-dichloro-3-fluorophenyl)ethoxy)-5-(1-(piperidin-4-yl)-1H-pyrazol-4-yl)pyridin-2-yl)acetamide;
(S)-3-(1-(2,6-dichloro-3-fluorophenyl)ethoxy)-5-(1-(1-methylpiperidin-4-yl)-1H-pyrazol-4-yl)pyridin-2-amine;
(S)-3-(1-(2,6-dichloro-3-fluorophenyl)ethoxy)-5-(1-(1-d₃-methyl-piperidin-4-yl)-1H-pyrazol-4-yl)pyridin-2-amine;
(S)-3-(1-(2,6-dichloro-3-fluorophenyl)ethoxy)-5-(1-(1-ethylpiperidin-4-yl)-1H-pyrazol-4-yl)pyridin-2-amine;
(S)-3-(1-(2,6-dichloro-3-fluorophenyl)ethoxy)-5-(1-(1-d₅-ethyl-piperidin-4-yl)-1H-pyrazol-4-yl)pyridin-2-amine;
(R)—N-(3-(1-(2,6-dichloro-3-fluorophenyl)ethoxy)-5-(1-(piperidin-4-yl)-1H-pyrazol-4-yl)pyridin-2-yl)acetamide;
(R)-3-(1-(2,6-dichloro-3-fluorophenyl)ethoxy)-5-(1-(1-d₃-methyl-piperidin-4-yl)-1H-pyrazol-4-yl)pyridin-2-amine;
(R)-3-(1-(2,6-dichloro-3-fluorophenyl)ethoxy)-5-(1-(1-d₅-ethyl-piperidin-4-yl)-1H-pyrazol-4-yl)pyridin-2-amine; and
(R)-3-(1-(2,6-dichloro-3-fluorophenyl)ethoxy)-5-(1-(1-ethylpiperidin-4-yl)-1H-pyrazol-4-yl)pyridin-2-amine.

19. A pharmaceutical composition comprising a compound of claim 14 or a pharmaceutically acceptable salt, solvate or a prodrug or an enantiomer thereof; and a pharmaceutically acceptable carrier.

20. The pharmaceutical composition of claim 19, further comprising an anti-neoplastic agent, an immunosuppressant, an immunostimulant, or combinations thereof.

21. A method of treating a disease or condition selected from hyper-proliferative disorder, ALK kinases mediated disorder, and neoplasia in a subject, the method comprising a step of administering to the subject an effective amount of a composition of claim 19.

22. The method of claim 21, wherein the disease or condition is hyper-proliferative disorder.

23. The method of claim 21, wherein the disease or condition is ALK kinases mediated disorder.

25. The method of claim 21, wherein the disease or condition is neoplasia.

26. The method of claim 21, further comprising a step of co-administering to the subject a second anti-cancer agent useful in the treatment of a disease or condition selected from hyper-proliferative disorder, ALK kinases mediated disorder, and neoplasia.

27. The method of claim 26, the second anti-cancer agent is useful in the treatment of neoplasia.
